(19) United States
(12) Patent Application Publication
(10) Pub. No.: US 2026/0147919 A1
(43) Pub. Date: May 28, 2026

(54) METHOD OF OBTAINING ELECTRONIC HEALTH RECORDS OF ANY PATIENT FROM ANY PROVIDER USING A GENERATIVE ARTIFICIAL INTELLIGENCE MODEL HAVING PERMISSION TO ACCESS THE ELECTRONIC HEALTH RECORDS WITH MINIMAL PATIENT INPUT

(71) Applicant: Kris Narayan, San Ramon, CA (US)

(72) Inventor: Kris Narayan, San Ramon, CA (US)

(21) Appl. No.: 19/177,844

(22) Filed: Apr. 14, 2025

Related U.S. Application Data
(60) Provisional application No. 63/732,506, filed on Aug. 23, 2024.

(51) Int. Cl. G06F 21/62; G16H 10/60

(52) U.S. Cl. CPC G06F 21/6245; G16H 10/60

(57) ABSTRACT

A computer-program product, system, and method of retrieving electronic health records (EHRs) of a patient including: requesting legally compliant permission from a patient to access a plurality of EHRs of the patient from a plurality of separate electronic storage devices storing at least one of the EHRs of the patient in a non-transitory computer-readable recording medium; receiving an access request from the patient via a natural language interface to retrieve the EHRs of the patient from the separate electronic storage devices; processing the access request using a generative artificial intelligence (AI) model having a unique licensing number assigned thereto; generating via the AI model a structured access request query; and sending via a communication network the structured access request query with the legally compliant permission to the separate electronic storage devices and thereby retrieving the EHRs.

### (B) CROSS-REFERENCE TO RELATED APPLICATIONS

The present application claims the benefit of U.S. Provisional Patent Application 63/732,506, filed Aug. 23, 2024, which is incorporated by reference herein.

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to the handling of electronic medical records (EMR) and electronic health records (EHR) and more particularly pertains to a new method for obtaining such records in a frictionless manner, that is, with minimal patient input. It should be noted that any reference in the present application to either EMRs or EHRs should be understood as referring to both EMRs and EHRs or each one interchangeably, though each type of record-keeping system is technically different and contains some different information. For convenience, only EHRs will be referred to in the following, but should be understood as also applying to EMRs.

Lifestyle factors contribute to the development of many chronic diseases, and their associated morbidity and mortality. Many of these chronic health conditions may be prevented, and an individual's span and quality of life improved through changes in lifestyle, such as consuming a healthy diet, exercising regularly and eliminating the abuse of tobacco and alcohol.

In particular, cigarette smoking is the single most preventable cause of premature death in the United States. More than 430,000 Americans die each year from smoking-related illness, translating to one in every five deaths. An additional 110,000 people die of causes related to alcohol abuse.

Obesity is another major cause of morbidity and mortality in the United States. More than half of all adults in the United States are considered overweight or obese. Far from being a purely cosmetic disorder, obesity substantially increases morbidity and impairs the quality of life in affected individuals. It is also a risk factor for chronic diseases including hypertension, coronary heart disease, Type II diabetes, gallbladder disease, osteoarthritis and cancers of the breast, colon, and uterus. Thus it is important to reduce body weight among obese and overweight individuals, and to prevent further weight gain in both normal and over-weight individuals.

Physical inactivity, defined as the absence of leisure time physical activity such as recreational exercise, is another major lifestyle-related risk factor for chronic health related conditions. In the United States and other developed countries, industrial automation has caused the majority of the population to be involved in sedentary occupations. Chronic conditions related to inactivity include coronary heart disease, hypertension, Type II diabetes, depression anxiety, osteoporotic hip fractures and obesity.

Lifestyle modifications alone successfully can be used to treat many chronic health conditions, without resorting to the usage of medication. For instance, mild hypertension is often controlled through lifestyle changes such as dietary modification, weight reduction, stress control, and physical activity. Weight control in itself delays and even prevents the onset of Type II Diabetes and mild hypertension.

In addition to lifestyle factors, the risk of developing many chronic health conditions is affected by a family history of disease. Diseases such as cancer, heart disease, elevated lipids, obesity, diabetes, stroke, high blood pressure, alcoholism, mental illness, and allergies all are affected.

EHRs started to be increasingly popular in 2004 when the U.S. created the Office of the National Coordinator of Information Technology with a plan to create EHRs for most Americans to share information privately and securely under their own authorization. While the implementation of this EHR has happened successfully and most health care providers have adopted this process, it has predominantly become a tool for physicians and hospitals and related organizations such as insurers to use to administer and process care. The most important part of this ecosystem, the patients, find the procurement process very difficult and cumbersome. For patient-consent-initiated requests, current schemes require the patient to specify substantial information, such as provider name, provider address, doctor or physician name, digital log in, user identification, and passwords, as well as personal data, such as name, address, date of birth, phone number, and social security number. Many EHR interfaces further require access confirmation responses, such as multiple screens asking the patient if the patient is certain the patient wishes to retrieve the EHR and/or provide it to another party. Such processes can be quite challenging for most anyone, especially the elderly or ill patients, as patients often forget one or pieces of information, answer an inquiry incorrectly, or are reluctant to provide personal information out of privacy concerns or fear of identity theft or other fraud.

The new method provides a scheme to overcome all such complex problems using a generative artificial intelligence (AI) model to obtain the EHR of any patient with only minimal and readily available information, such as name, address and date of birth. The method further includes interaction with the generative AI to receive information and analysis of the EHR, as well as other health data, such as social determinants of health (SDOH) data and recorded physiological functions from smart devices and wearables, to form an overall health report or summary and related advice and information.

### (2) Description of Related Art Including Information Disclosed Under 37 CFR 1.97 and 1.98

The prior art relates to EHR retrieval systems and methods. The prior art, as best understood, does not disclose a method and related software application that enables patients to acquire EHRs to access a plurality of EHRs of the patient from a plurality of separate electronic storage devices storing at least one of the EHRs of the patient in a non-transitory computer-readable recording medium, wherein a generative artificial intelligence (AI) model having a unique licensing number assigned thereto receives and processes an access query from the patient, generates a structured access request query, and then sends via a communication network the structured access request query with a legally compliant permission from the patient to the separate electronic storage devices and thereby retrieves the EHRs.

### (h) BRIEF SUMMARY OF THE INVENTION

An embodiment of the disclosure meets the needs presented above in a computer-program product tangibly embodied in a non-transitory computer-readable recording medium in an electronic device for retrieving electronic health records (EHRs) of a patient. The non-transitory computer-readable recording medium stores one or more programs which when executed by a hardware processor cause the non-transitory recording medium to perform steps including: requesting legally compliant permission from a patient to access a plurality of EHRs of the patient from a plurality of separate electronic storage devices storing at least one of the EHRs of the patient in a non-transitory computer-readable recording medium; receiving an access request from the patient via a natural language interface to retrieve the EHRs of the patient from the separate electronic storage devices; processing the access request using a generative artificial intelligence (AI) model having a unique licensing number assigned thereto; generating via the AI model a structured access request query; and sending via a communication network the structured access request query with the legally compliant permission to the separate electronic storage devices and thereby retrieving the EHRs.

The method facilitates procurement of EHRs of any patient treated by any provider, such as a doctor, physician, health coach, clinic, hospital, laboratory, or medical facility, and whose EHRs are held by any company and accessible with minimal information, such as patient name, address and date of birth using a generative AI model or coach. The generative AI model also will utilize a unique licensing number, such as, for example, a National Provider Identifier (NPI) provided by the Centers for Medicare and Medicaid Services (CMS) of the U.S. federal government, to facilitate such request and is augmented by a clinical supervisor for spot checks to ensure accuracy and validity of such operations. The method and related software application will help individuals understand their health status and their areas of concerns, as well as provide them a wealth of resources to understand and improve their health to become happy, healthy individuals, as opposed to simply receiving raw data in their EHRs that is difficult if not impossible to utilize in any practical or useful manner.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a flowchart illustrating a method of mapping patient data, according to some embodiments of the present disclosure.

FIG. 2 is a block diagram illustrating a system, according to some embodiments of the present disclosure.

FIG. 3 is a block diagram further illustrating the system from FIG. 2, according to some embodiments of the present disclosure.

FIG. 4 is a block diagram further illustrating the system from FIG. 2, according to some embodiments of the present disclosure.

FIG. 5 is a block diagram illustrating a computer program product, according to some embodiments of the present disclosure.

FIG. 6 illustrates an aspect of the subject matter in accordance with one embodiment.

FIG. 7 illustrates an aspect of the subject matter in accordance with one embodiment.

FIG. 8 illustrates an aspect of the subject matter in accordance with one embodiment.

FIG. 9 illustrates an aspect of the subject matter in accordance with one embodiment.

FIG. 10 illustrates an aspect of the subject matter in accordance with one embodiment.

FIG. 11 illustrates an aspect of the subject matter in accordance with one embodiment.

FIG. 12 illustrates an aspect of the subject matter in accordance with one embodiment.

FIG. 13 is a diagram of a EHR retrieval system according to an embodiment of the disclosure.

FIG. 14 is a diagram of a EHR retrieval system according to an embodiment of the disclosure.

FIG. 15 is a flowchart of an embodiment of the disclosure.

FIG. 16 is a flowchart of an embodiment of the disclosure.

FIG. 17 is a flowchart of an embodiment of the disclosure.

### (j) DETAILED DESCRIPTION OF THE INVENTION

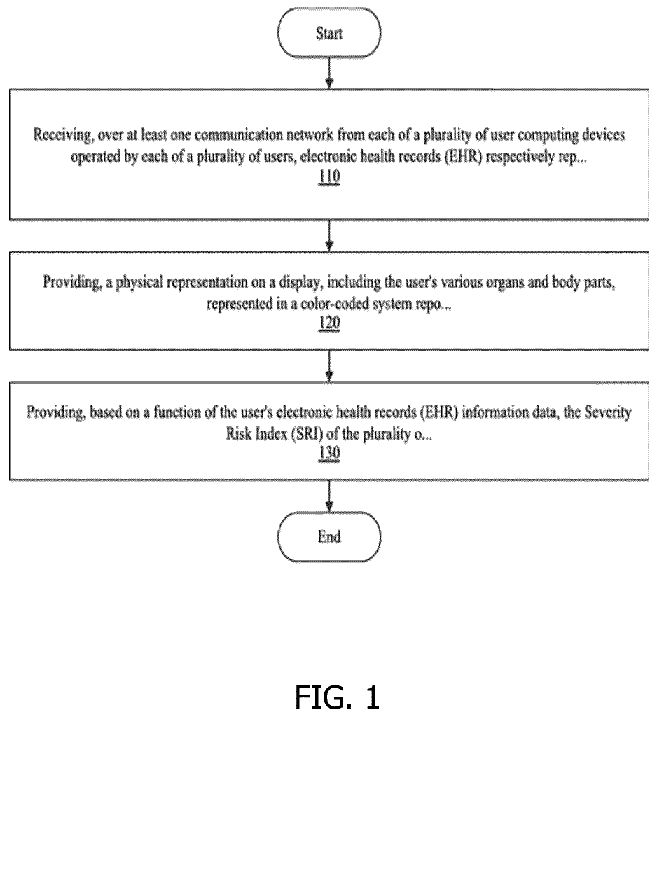

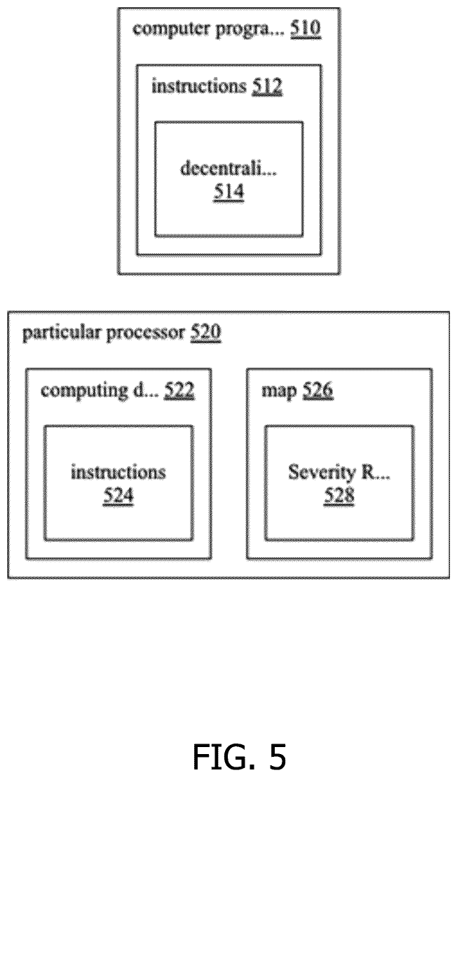

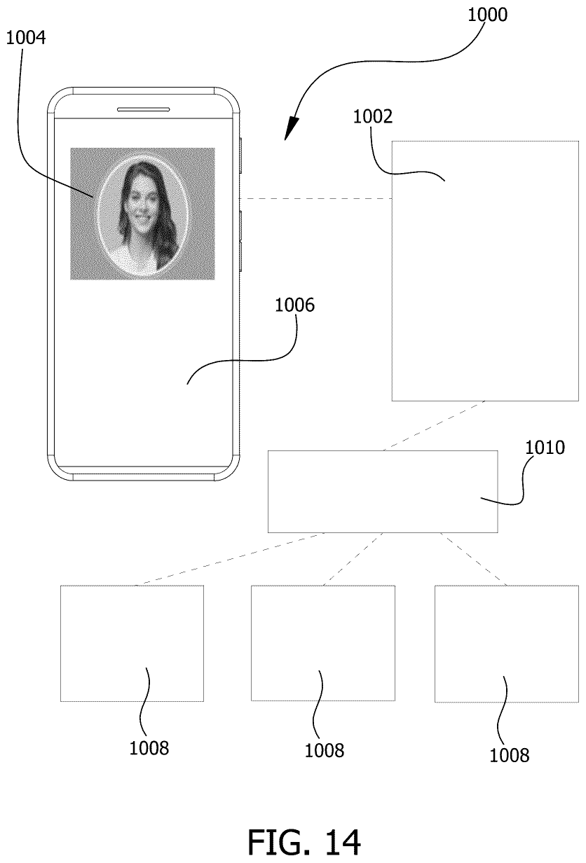

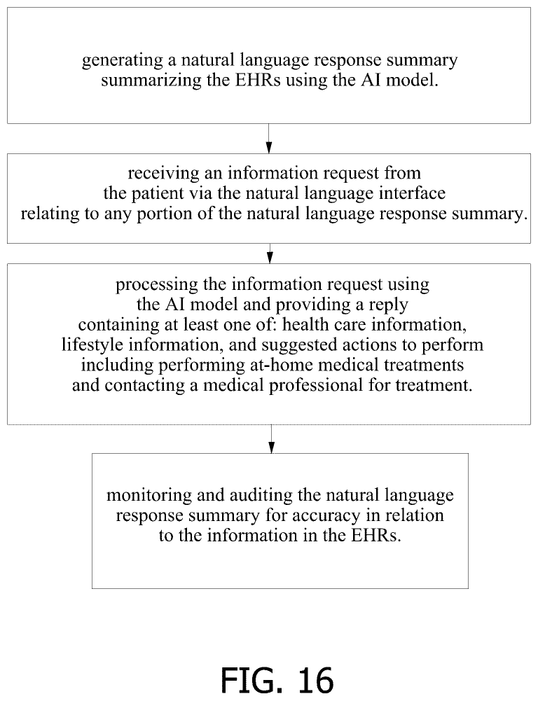

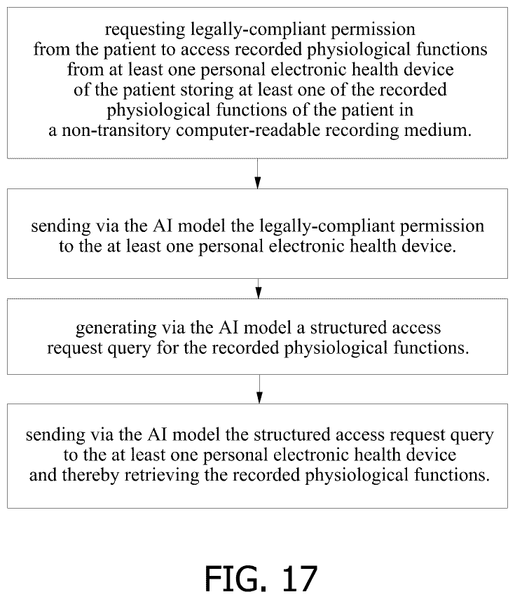

With reference now to the drawings, and in particular to FIGS. 1 through 17 thereof, a new EHR retrieval system embodying the principles and concepts of an embodiment of the disclosure and generally designated by the reference numeral 1000 will be described.

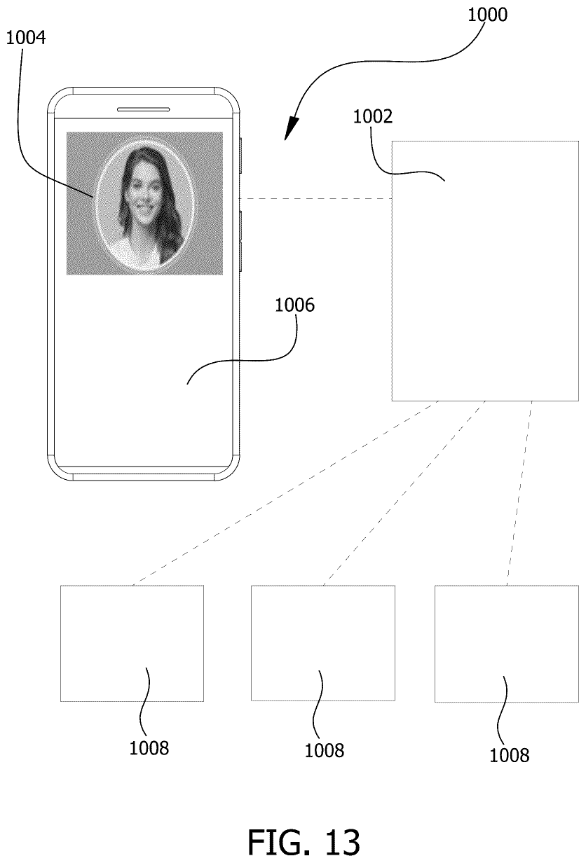

As best illustrated in FIGS. 13 through 17, the EHR retrieval system 1000 generally comprises a computer-program product 1002, a generative artificial intelligence (AI) model 1004, a personal electronic device 1006, and a plurality of separate electronic storage devices 1008. The personal electronic device 1006 could be a mobile phone, smart phone, laptop, tablet computer, or other such device. The personal electronic device 1006 is designed to display an interface for a user to interact with the computer-program product 1002. The separate electronic storage devices 1008 are designed to store EHRs in a non-transitory computer-readable recording medium, as is well known in the healthcare industry. These different components can communicate over a standard network that has wired and wireless capabilities, which is not shown in any detail.

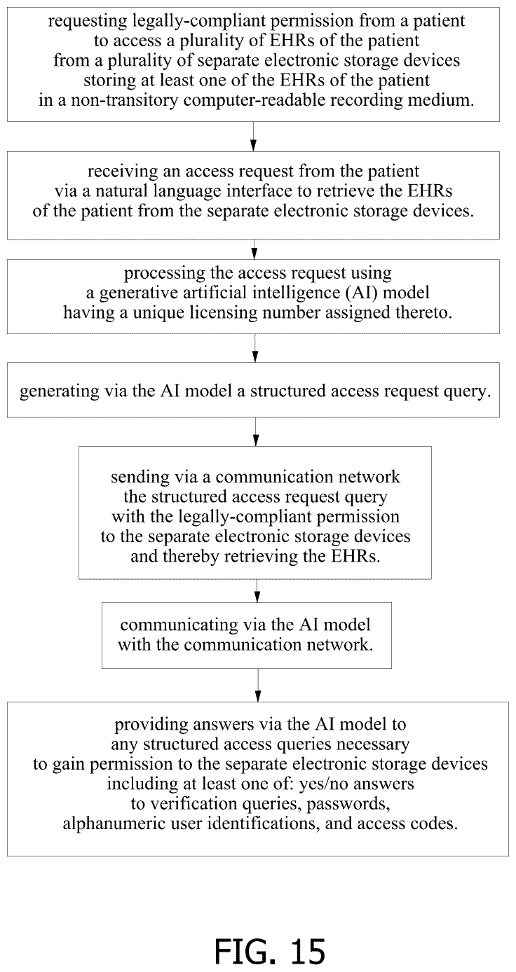

The computer-program product 1002 is tangibly embodied in a non-transitory computer-readable recording medium in an electronic device for retrieving electronic health records (EHRs) of a patient. The non-transitory computer-readable recording medium stores one or more programs which when executed by a hardware processor cause the non-transitory recording medium to perform a number of steps, as described in the following and shown in part in the flow charts in FIGS. 15 through 17. The computer-program product 1002 requests legally compliant permission from a patient to access a plurality of EHRs of the patient from a plurality of separate electronic storage devices 1008 storing at least one of the EHRs of the patient in a non-transitory computer-readable recording medium. What is meant by “legally compliant permission” is permission that complies with laws governing access to the EHRs of the patient in the jurisdiction of the patient, such as state and/or federal or national laws and regulations, such as HIPAA (Health Insurance Portability and Accountability Act) in the U.S. Once this legally compliant permission is received and stored by the computer-program product 1002, the computer-program product 1002 can receive an access request from the patient via a natural language interface to retrieve the EHRs of the patient from the separate electronic storage devices 1008. Such access request can come from a patient checking off consent boxes in the natural language interface to request EMR records. Such consent requests can be reviewed and approved by an auditing clinician or client health system's supervising physician as needed The access request is processed using the generative artificial intelligence (AI) model 1004, which has a unique licensing number assigned thereto. The licensing number, as discussed herein, authorizes the release of EHRs. The AI model 1004 then generates a structured access request query in order to access the EHRs. The structured access request query, along with the legally compliant permission, is sent via the communication network to the separate electronic storage devices 1008 to thereby retrieve the EHRs.

In one possible embodiment, the AI model 1004 communicates with the communication network and provides answers to any structured access queries necessary to gain permission to the separate electronic storage devices 1008, which structured access queries include at least one of: yes/no answers to verification queries, passwords, alphanumeric user identifications, and access codes. Many systems that store EHRs require additional individual verification queries, and the AI model 1004 is designed to answer those queries on behalf of patient in order to access the EHRs. In another possible embodiment, the AI model 1004 communicates with an EHR retrieval service or broker 1010 that is authorized to retrieve multiple EHRs from multiple providers. Additional security measures can include encryption methods for data at rest and in transit, secure authentication mechanisms for both patients and healthcare providers, and compliance with relevant data protection laws (e.g., GDPR, HIPAA). Specifying measures for data access control, such as role-based permissions or audit logs, would reinforce trust in the system's ability to protect patient information.

In one possible embodiment, the unique licensing number is a ten-digit National Provider Identifier (NPI) number. The NPI number is a Health Insurance Portability and Accountability Act (HIPAA) Administrative Simplification Standard in the U.S. The NPI is a unique identification number for covered health care providers, which can provide access to EHRs and EMRs.

In the exemplary embodiment, the legally compliant permission includes only a name including a current name and any previous names, address information, such as a ZIP code or postal code or street number, for example, and a birth date of the patient. This minimal information allows a patient to easily grant access to the patients EMRs in a frictionless manner without the need for multiple steps from multiple applications that require a great variety of information, such as passwords, user IDs, and names of providers and physicians. In another possible embodiment, additional well known data could also be required for the legally compliant permission, such as social security number or telephone number.

As discussed herein, the computer-program product 1002 uses the AI model 1004 to generate a natural language response summary summarizing the EHRs using the AI model 1004. The computer-program product 1002 can receive an information request from the patient via the natural language interface relating to any portion of the natural language response summary. The computer-program product 1002 then uses the AI model 1004 to process the information request and provide a reply containing at least one of: health care information, lifestyle information, and suggested actions to perform includes performing at-home medical treatments and contacting a medical professional for treatment. Such AI suggestions of medical actions or lifestyle changes could be accompanied by information for a patient to understand the reasoning behind those recommendations. The AI model could also explain its decision-making process—such as referencing specific data points from the patient's health records or evidence-based guidelines—which would increase transparency and patient confidence in the system. The AI model could also provide patients with the option to request clarifications on specific health recommendations, which would further enhance the user experience and facilitate better decision-making. Such monitoring and auditing the AI-generated health summaries can provide a dynamic feedback loop for improving the system's responses over time. This could involve collecting feedback from patients, medical professionals, or other relevant stakeholders to refine the AI model's suggestions. Integrating machine learning techniques that allow the system to adapt and improve based on new health data or evolving medical research could enhance the accuracy and relevance of the health reports. In other words, the AI model can utilize multiple iterations of the patient providing an information request and then processing that request in view of the natural language response summary.

In at least one possible embodiment, a physician or other medical professional can monitor and audit the natural language response summary for accuracy in relation to the information in the EHRs. The AI model 1004 can then be further trained to provide more and more accurate information customized to an individual patient.

In one possible embodiment, the computer-program product 1002 requests legally compliant permission from the patient to access recorded physiological functions from at least one personal electronic health device of the patient storing at least one of the recorded physiological functions of the patient in a non-transitory computer-readable recording medium. As discussed herein, the personal electronic health device could be a smart device or a wearable device, such as heart monitor, smart watch, step tracker, or other similar device capable of recording and transmitting physiological functions of the patient. The computer-program product 1002 sends via the AI model 1004 the legally compliant permission to the at least one personal electronic health device. The computer-program product 1002 then generates via the AI model 1004 a structured access request query for the recorded physiological functions. The computer-program product 1002 then sends via the AI model 1004 the structured access request query to the at least one personal electronic health device and thereby retrieves the recorded physiological functions. The computer-program product 1002 then combines and evaluating the EHRs and the recorded physiological functions and generates a natural language overall health report of the patient using the AI model 1004 containing a variety of health and personal information, such as: medical status of patient organs and body parts; comparison of the patient to other patients in a general patient database having similar personal characteristics; a list of medical personnel to assist with patient health conditions; a list of medical organizations to assist with patient health conditions; a list of medical services or products to assist with patient health conditions; a list of coaches or advisers to assist with patient health conditions; a list of support groups or networks having persons with similar patient health conditions to assist with patient health conditions; and a list of non-professional personal contacts to assist with patient health conditions.

As discussed herein, the computer-program product 1002 can generate an image of a human body with the predetermined organs and body parts represented on the image. The computer-program product 1002 then analyzes the EHRs and assigns selected data to a respective one of predetermined organs and body parts of the patient. The computer-program product 1002 then maps the selected data to a respective one of the predetermined organs and body parts. The computer-program product 1002 presents the image of the human body with the selected data adjacent the predetermined organs and body parts on the personal electronic device 1006 of the patient. In one possible embodiment, the computer-program product 1002 can then analyze the EHRs and create an overall health score based for the patient represented by a numerical value in a predetermined range using health risk factors to adjust the overall health score. The computer-program product 1002 presents the overall health score on the personal electronic device 1006 of the patient. Such user interface (UI) design can customize options, particularly in terms of how the patient views and interacts with their health information. Example: will the system allow patients to adjust how EHR data and health reports are displayed, such as choosing between detailed medical terminology or simplified explanations? Including features like multi-language support or options for patients with different levels of health literacy would ensure broader accessibility. Offering visual aids like graphs, charts, or timelines to track health trends could improve patient engagement and understanding.

In the following description, a source of content is understood to include a record of the content, such as an HTML file and a server, respectively, to provide non-limiting examples. Personalized health content is understood to refer generally to health content that is personalized to an individual's profile and general health attributes, and not merely to a health situation of a generic individual having a given health related condition. In a system for delivering health information to, and monitoring a plurality of individuals, personalized health content is understood to refer to health content customized to each individual. The statement that an input device is in communication with some data processing means is understood to mean that the data processing means is adapted to use data specified by the input either directly or indirectly. The term server is understood to refer to an information-generating device capable of communicating with a plurality of clients; servers include computer servers and television delivery systems. The term individual is understood to refer to a person at risk for engaging in behavior having adverse health consequences, as well as a person suffering from a chronic condition or disease.

An exemplary embodiment discussed below describes a computer-based implementation of a system and method for general personal health improvement with special emphasis on physical activity, diet and nutrition. It will be clear to an individual with ordinary skill in the art that the present invention is suitable for preventive care directed to many other health conditions. Moreover, there are many well-known structures, interfaces and processes that are suitable for implementing the present invention.

FIG. 1 is a flowchart that describes a method of mapping patient data, according to some embodiments of the present disclosure. In some embodiments, at 110, the method may include receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners.

In some embodiments, at 120, the method may include providing a physical representation on a display, including the user's various organs and body parts, represented in a color-coded system reporting the related electronic health records (EHR) data representing a plurality of medical records laboratory results. At 130, the method may include providing, based on a function of the user's electronic health records (EHR) information data, the Severity Risk Index (SRI) of the plurality of medical records, and the medical records laboratory results, the user's customized overall health score. Mapping, on the display of the user's whole-body avatar, the user's electronic health records (EHR) information data into a plurality of visual representations comprising a Severity Risk Index (SRI) representing the risk of a plurality of ailments mapped over the user's plurality of organs and body parts.

In some embodiments, the method may include creating a user's whole body avatar by using an image-capturing device or picture file uploaded to the communication network, which may be aggregated with the electronic health records (EHR) data. In some embodiments, the method may include providing further additional hidden details about the Severity Risk Index (SRI) through a clickable navigation link to data attached to the visual representation.

In some embodiments, the method may include providing a history of the user's customized overall health score on the display. In some embodiments, the method may include providing a plurality of score drivers. The score drivers may comprise factors representing user-controlled parameters, activities, behaviors, and lifestyles that affect the overall health score may be derived. In some embodiments, the electronic health records (EHR) include information representing at least one of name, DNA, birthdate, gender, age, height, weight, heart rate, blood pressure, blood type, and blood glucose.

In some embodiments, the method may include providing a connection to a social media network through the communication network, to present to the user at least one of the following, the method may include performing one or more additional steps. Medical status of the user's organ.

Comparison of the user's status compared to other user's status in the same age group or demographic. A list of caregivers or physicians that can help with such an ailment. A list of services or products that can help with an ailment. A list of health care coaches that can educate the user about an ailment. A list of influencers that may be knowledgeable in such health topics. A network of other users with similar ailments. Or connection to the user's family, friends or classmates from user's local health club or gym. In some embodiments, the communication network may provide a virtual assistant embedded within the user interface and connected to the communication network to interact with the user to provide voice-driven navigation of the electronic health records (EHP) using natural language processing (NLP). In some embodiments, the electronic health records (EHR) databases may be web 3.0 compliant.

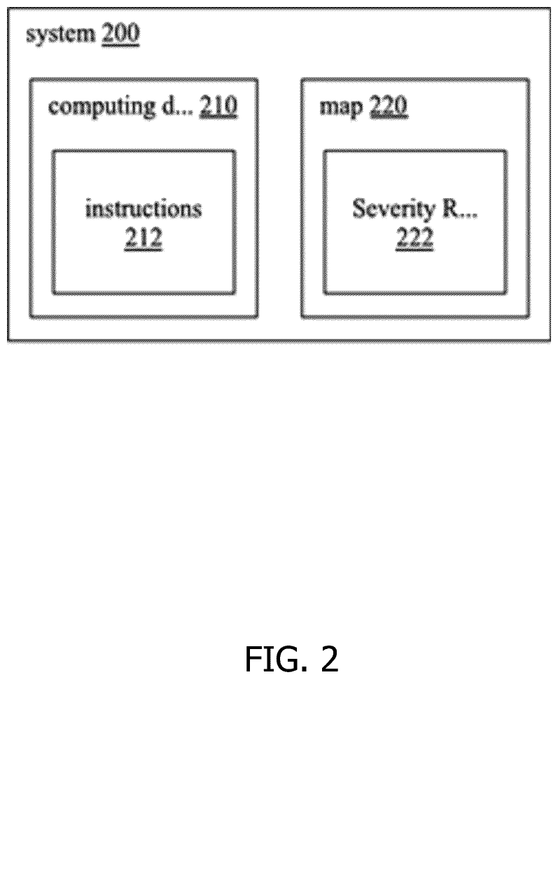

\FIG. 2 is a block diagram that describes a system 200, according to some embodiments of the present disclosure. In some embodiments, the system 200 may include a computing device 210 configured to access non-transitory processor-readable media. The system 200 may also include map 220, on the display of the user's whole-body avatar, the user's electronic health records (EHR) information data into a plurality of visual representations. The computing device 210 may also include instructions 212 that, when executed by the computing device 210 cause the computing device 210 to derive a customized overall health score.

In some embodiments, the method may include receiving, over at least one communication network from each of a plurality of user computing devices operated by each of a plurality of users, electronic health records (EHR), respectively representing information from the plurality of user's electronic medical records, procured from the user's health care provider or the user's electronic health records (EHR) partners. In some embodiments, the method may include providing a physical representation on a display.

In some embodiments, the user's various organs and body parts, represented in a color-coded system reporting the related electronic health records (EHR) data representing a plurality of medical records laboratory results. The map 220 may include a Severity Risk Index 222 (SRI) representing the risk of a plurality of ailments mapped over the user's plurality of organs and body parts. And provide, based on a function of the user's electronic health records (EHR) data, the Severity Risk Index 222 (SRI) of the plurality of medical records, and the plurality of medical records laboratory results, the user's customized overall health score.

In some embodiments, the user's whole-body avatar may be created by using an image-capturing device or picture file uploaded to the communication network, which may be aggregated with the electronic health records (EHR) data. In some embodiments, the user's electronic health records (EHR) provide further additional hidden details about the Severity Risk Index 222 (SRI) through a clickable navigation link to data attached to the visual representation.

In some embodiments, the processor may be configured to execute instructions that cause the processor to provide a history of the customized overall health score. In some embodiments, the processor may be configured to execute instructions that cause the processor to provide a plurality of score drivers. The score drivers may also include factors representing user-controlled parameters, activities, behaviors, and lifestyles that affect the overall health score may be derived.

In some embodiments, the electronic health records (EHR). In some embodiments, the communication network may provide a virtual assistant embedded within the user interface to interact with the user to provide voice-driven navigation of the electronic health records (EHP) using natural language processing (NLP). In some embodiments, the electronic health records (EHR) databases may be web 3.0 compliant.

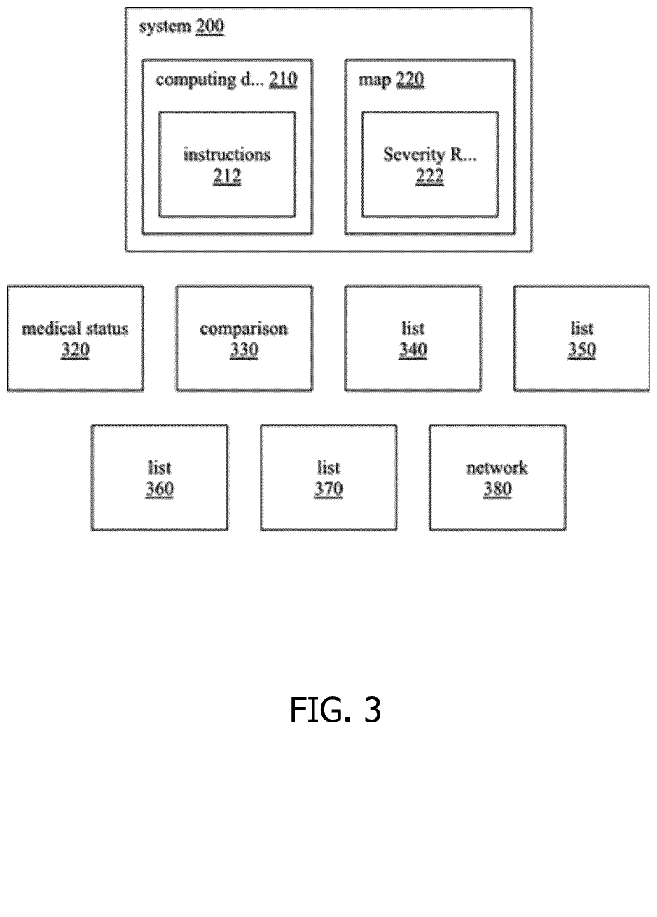

FIG. 3 is a block diagram that further describes the system 200 from FIG. 2, according to some embodiments of the present disclosure. In some embodiments, the communication network may provide a connection to a social media network through the communication network, presents to the user at least one of the following: connection to the user's family, friends or classmates from user's local health club or gym.

FIG. 3 is a block diagram that further describes the system 200 from FIG. 2, according to some embodiments of the present disclosure. In some embodiments, the communication network may provide a connection to a social media network through the communication network, presenting to the user at least one of the following: connection to the user's family, friends or classmates from user's local health club or gym.

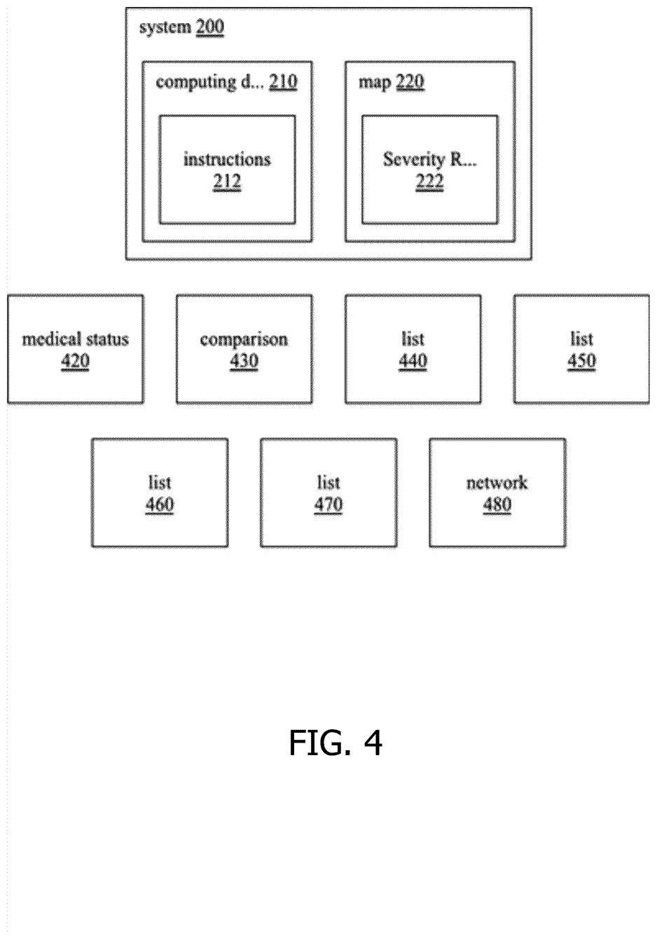

FIG. 4 is a block diagram that further describes the system 200 from FIG. 2, according to some embodiments of the present disclosure. In some embodiments, the communication network may provide a connection to a social media network through the communication network, presenting to the user at least one of the following: connection to the user's family, friends or classmates from user's local health club or gym.

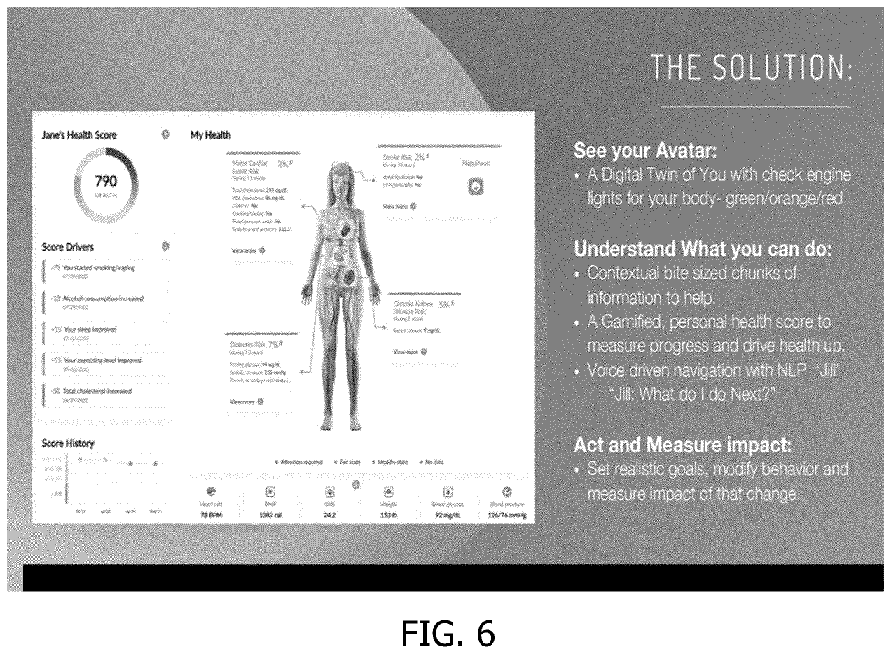

FIG. 6 is a view of the dashboard that displays an avatar of the patient mobile application dashboard that displays the patient's electronic health records (EHR) data aggregated to advise the patient of severity of any ailments, specific organ and body parts status and the patient's customized overall health score.

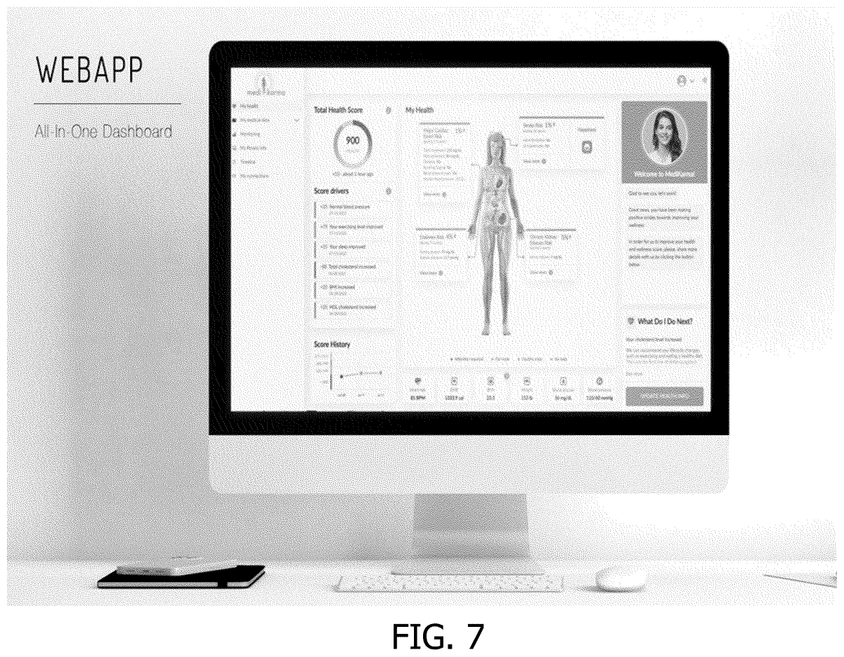

FIG. 7 is a view of the all in one dashboard on a computer display. FIG. 7 also depicts a rendering of “Jill” the virtual assistant that assists with advising the patient of next steps and answers any questions about the electronic health records data displaying the severity risk indices. Each of the severity risk indices are specific to different organs and body parts.

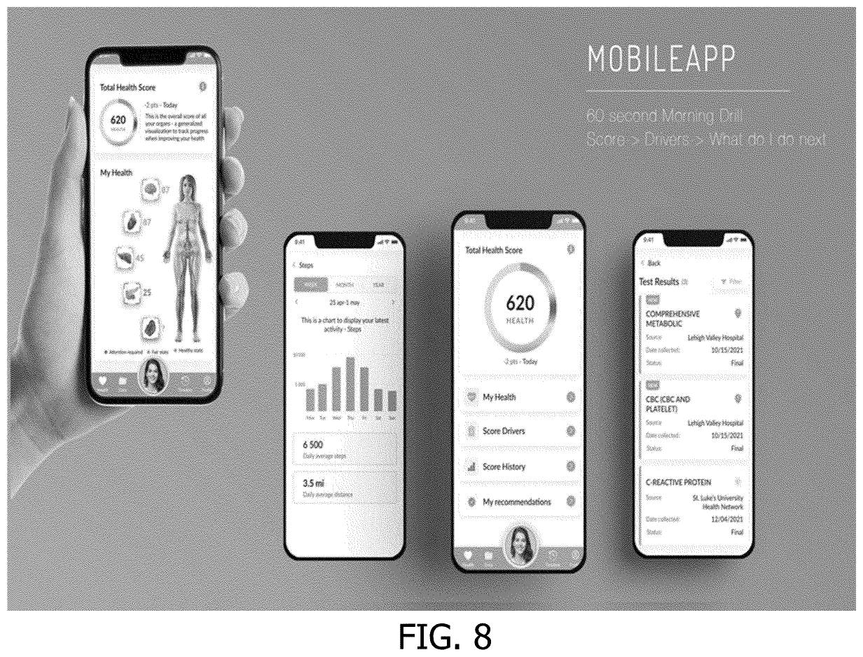

FIG. 8 is a view of the mobile application with various views of electronic health records data being displayed on a mobile device as the application would be used by the patient.

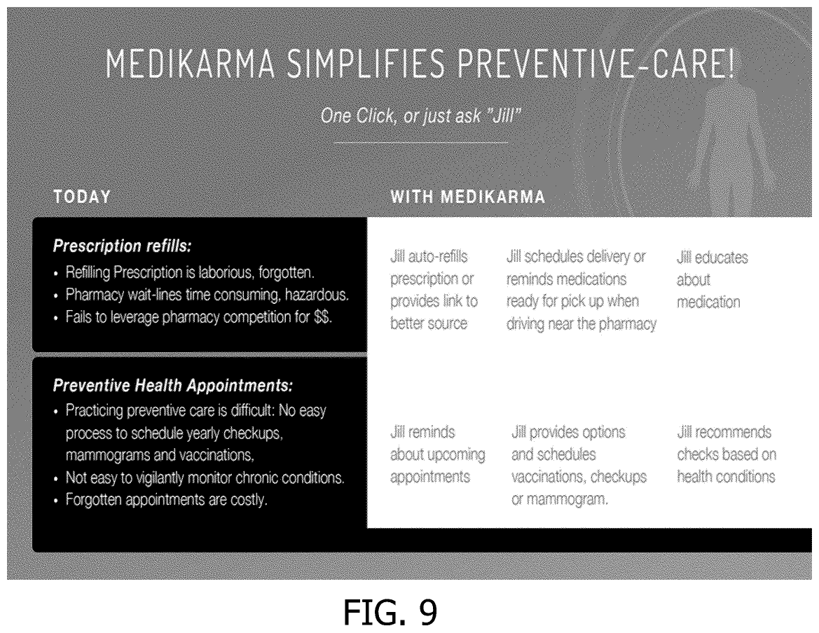

FIG. 9 is a view of the steps of a virtual assistant algorithm providing advice to the patient on the prescription refills and provides a link to better sources, if they exist. The virtual assistant educates the patient about the medication and reminds the patient of upcoming appointments, vaccinations, checkups or mammogram. Virtual Assistant “Jill” will make recommendations that would be in the best interest of the patient.

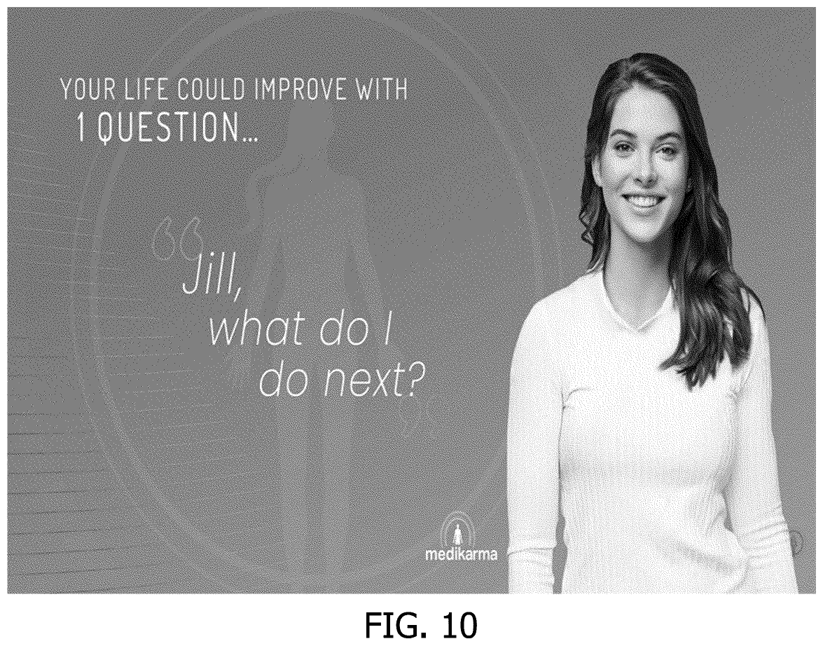

FIG. 10 is a view a hypothetical avatar of the Virtual Assistant “Jill”. Jill, like other Virtual Assistants that use Natural Language Processing, interacts with the patient to ensure a simple response to help ensure that the patient is able to understand the information.

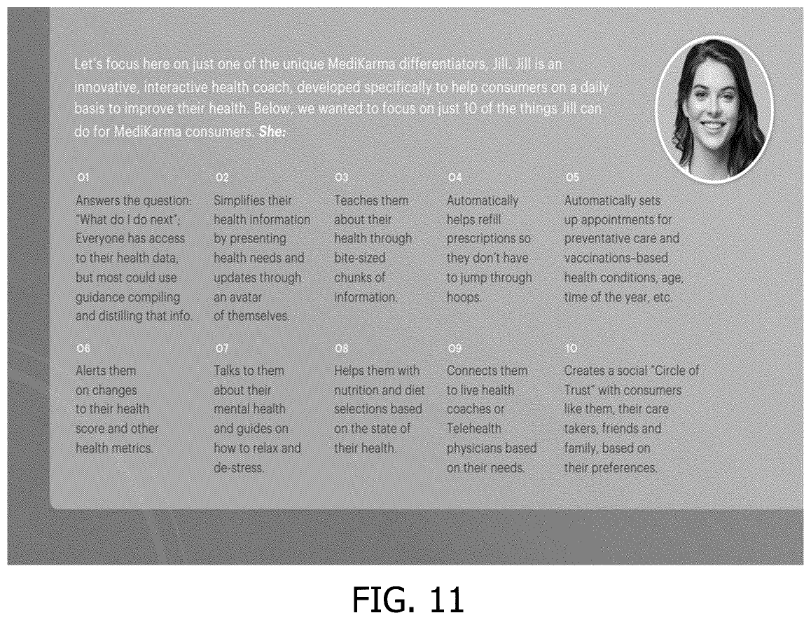

FIG. 11 is a view of the various tasks that the Virtual Assistant “Jill” can perform for the patient to facilitate the translation of the electronic medical data (EHR) and the customized overall health score.

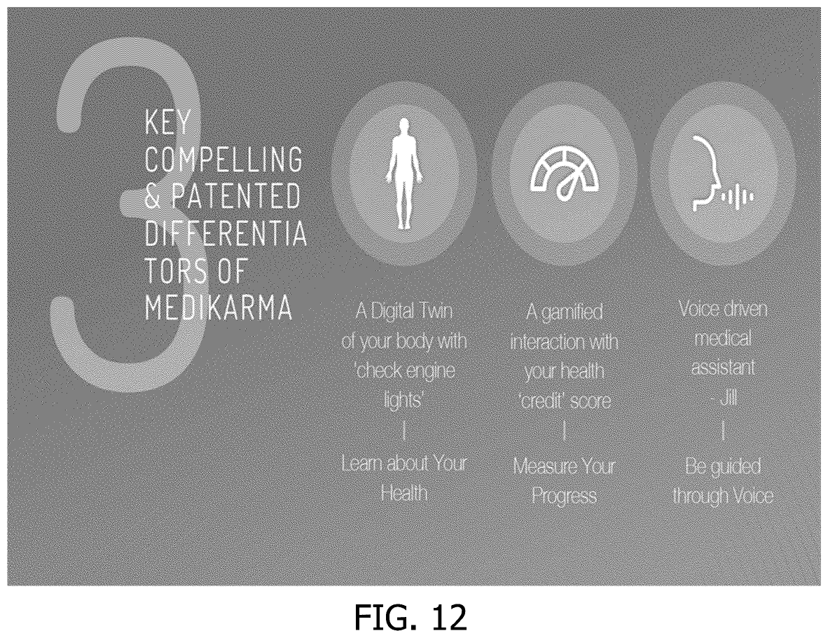

FIG. 12 is a description of the three differentiators from the claimed invention. Medical records are analyzed and aggregated to provide a digital twin of a patient's body with “check engine lights” to educate the patient about their health. A social media platform is connected to the application to create a gamified interaction with the patient's customized overall health score to know the status of any ailments the patient may be suffering to help the patient measure their progress. The patient interacts with the virtual assistant “Jill” and it provides guidance on the patient's health progress.

It will be clear to one skilled in the art that the above invention may be altered in many ways without departing from the scope of the invention. Various relative arrangements of inputs and processing means are possible for generating personalized health content. In addition to general health improvement, various diseases and behaviors are amenable to preventive care according to a method of the present invention, including asthma, hypertension, cardio-vascular disease, eating disorders, breathing disorders, blood pressure disorders, heart conditions, HIV, mental health dis-orders, smoking, and drug or alcohol abuse.

In one possible embodiment, the method involves using a software application, wherein upon a patient or provider's request for EMR of such patient, the generative AI model requests full name, address, and date of birth of the patient and full consent to procure, integrate and debug EMR data into the software application platform. The software application reviews such request, authenticates it with an AI check and spot checks by supervising clinician and submits such request to a third party responsible for providing EMRs. The third party returns the EMR and the software application processes such EMR along with integration of any known data from such patients' smart devices and wearables providing additional health information and any other SDOH information software application may have about this patient to form a unique longitudinal data for such patients.

In one possible embodiment, the software application uses such longitudinal data to provide any of the following: medical status of their organs and body parts; comparison of their status to their age group or demographics; a list of care givers or physicians that can help with such ailments; a list of services or products that can help; a list of health care coaches that can educate the individual; a list of influencers that are knowledgeable in such topics; a network of other individuals with similar ailments; and connection to personal contacts such as family, friends or classmates from a local health club.

In one possible embodiment, the software application facilitates visual representation of health care conditions overlaid over a patient's digital twin image. This process also enables elaborate access to health care summary and history of such individual derived from their EHRs and EMRs. Such elaborate access will involve information being displayed in panels surrounding the human body representation and can include information, including test results and procedures performed, to help individuals fully understand their entire medical history. The software application provides comprehensive longitudinal patient data integrated from the EHR and EMR, smart devices, and wearables on a single platform, accessible with a simple voice command. Unlike individual providers like Sutter or Kaiser, which only provide data for their respective hospitals, the software application consolidates EMRs and EHRs across multiple hospitals, clinics, and providers into one unified platform. The patient can retrieve any health data effortlessly using a natural language interface with the generative AI model. For example, the generative AI model could be an image of a person named Jill, and the patient could ask a question, such as, “Hey Jill, who was the doctor I visited last year when I had back pain?” The generative AI model will then provide that information in a conversational manner with any related analysis and suggestions of actions that could be taken by the patient.

Embodiments of the present disclosure may include a method of mapping patient data and representing data from electronic health records of an individual through a pictorial representation of their human body including receiving, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners.

Embodiments may also include providing, a physical representation on a display, including the user's various organs and body parts, represented in a color-coded system reporting the related electronic health records (EHR) data representing a plurality of medical records laboratory results. Embodiments may also include mapping, on the display of the user's whole body avatar, the user's electronic health records (EHR) information data into a plurality of visual representations including a Severity Risk Index (SRI) representing the risk of a plurality of ailments mapped over the user's plurality of organs and body parts. Embodiments may also include providing, based on a function of the user's electronic health records (EHR) information data, the Severity Risk Index (SRI) of the plurality of medical records, and the medical records laboratory results, the user's customized overall health score.

In some embodiments, the method, creating a user's whole body avatar by using an image-capturing device or picture file uploaded to the communication network, which may be aggregated with the electronic health records (EHR) data. In some embodiments, the method, providing further additional hidden details about the Severity Risk Index (SRI) through a clickable navigation link to data attached to the visual representation.

In some embodiments, the method, providing a history of the user's customized overall health score on the display. In some embodiments, the method, providing a plurality of score drivers. In some embodiments, the score drivers may include factors representing user-controlled parameters, activities, behaviors, and lifestyles that affect the overall health score may be derived.

In some embodiments, the electronic health records (EHR) includes information representing at least one of name, DNA, birthdate, gender, age, height, weight, heart rate, blood pressure, blood type, and blood glucose. In some embodiments, the method, providing a connection to a social media network through the communication network, to present to the user at least one of the following medical status of the user's organ. Embodiments may also include comparison of the user's status compared to other user's status in the same age group or demographic. Embodiments may also include a list of caregivers or physicians that can help with such an ailment.

Embodiments may also include a list of services or products that can help with an ailment. Embodiments may also include a list of health care coaches that can educate the user about an ailment. Embodiments may also include a list of influencers that may be knowledgeable in such health topics. Embodiments may also include a network of other users with similar ailments. Embodiments may also include or connection to the user's family, friends or classmates from user's local health club or gym.

In some embodiments, the communication network provides a virtual assistant embedded within the user interface and connected to the communication network to interact with the user to provide voice-driven navigation of the electronic health records (EHP) using natural language processing (NLP). In some embodiments, the electronic health records (EHR) databases may be web 3.0 compliant.

Embodiments of the present disclosure may also include a system including a computing device configured to access non-transitory processor-readable media having instructions that, when executed by the computing device cause the computing device to receive, over at least one communication network from each of a plurality of user computing devices operated by each of a plurality of users, electronic health records (EHR), respectively representing information from the plurality of user's electronic medical records, procured from the user's health care provider or the user's electronic health records (EHR) partners.

Embodiments may also include providing a physical representation on a display, including the user's various organs and body parts, represented in a color-coded system reporting the related electronic health records (EHR) data representing a plurality of medical records laboratory results. Embodiments may also include map, on the display of the user's whole-body avatar, the user's electronic health records (EHR) information data into a plurality of visual representations including a Severity Risk Index (SRI) representing the risk of a plurality of ailments mapped over the user's plurality of organs and body parts. Embodiments may also include and provide, based on a function of the user's electronic health records (EHR) data, the Severity Risk Index (SRI) of the plurality of medical records, and the plurality of medical records laboratory results, the user's customized overall health score.

In some embodiments, the user's whole-body avatar may be created by using an image-capturing device or picture file uploaded to the communication network, which may be aggregated with the electronic health records (EHR) data. In some embodiments, the user's electronic health records (EHR) provide further additional hidden details about the Severity Risk Index (SRI) through a clickable navigation link to data attached to the visual representation.

In some embodiments, the processor may be configured to execute instructions that cause the processor to provide a history of the customized overall health score. In some embodiments, the processor may be configured to execute instructions that cause the processor to provide a plurality of score drivers. In some embodiments, the score drivers may include factors representing user-controlled parameters, activities, behaviors, and lifestyles that affect the overall health score may be derived.

In some embodiments, the electronic health records (EHR) include information representing at least one of: name, DNA, birthdate, gender, age, height, weight, heart rate, blood pressure, blood type, and blood glucose. In some embodiments, the communication network provides a connection to a social media network through the communication network, presenting to the user at least one of the following medical status of the user's organ. Embodiments may also include comparison of the user's status compared to other user's status in the same age group or demographic. Embodiments may also include a list of caregivers or physicians that can help with such an ailment. Such comparison process could enhance the clarity and transparency of these evaluations by specifying factors or criteria the AI model uses to perform comparisons, such as age, medical history, lifestyle habits, or genetic information. Such comparisons or recommendations can be based on relevant and anonymized datasets, respecting patient privacy while ensuring that the comparisons are meaningful and actionable.

Embodiments may also include a list of services or products that can help with an ailment. Embodiments may also include a list of health care coaches that can educate the user about an ailment. Embodiments may also include a list of influencers that may be knowledgeable in such health topics. Embodiments may also include a network of other users with similar ailments. Embodiments may also include or connection to the user's family, friends or classmates from user's local health club or gym.

In some embodiments, the communication network provides a connection to a social media network through the communication network, presents to the user at least one of the following medical status of the user's organ. Embodiments may also include comparison of the user's status compared to other user's status in the same age group or demographic. Embodiments may also include a list of caregivers or physicians that can help with such an ailment.

Embodiments may also include a list of services or products that can help with an ailment. Embodiments may also include a list of health care coaches that can educate the user about an ailment. Embodiments may also include a list of influencers that may be knowledgeable in such health topics. Embodiments may also include a network of other users with similar ailments. Embodiments may also include or connection to the user's family, friends or classmates from user's local health club or gym. In some embodiments, the communication network provides a virtual assistant embedded within the user interface to interact with the user to provide voice-driven navigation of the electronic health records (EHP) using natural language processing (NLP). In some embodiments, the electronic health records (EHR) databases may be web 3.0 compliant.

Embodiments of the present disclosure may also include a computer program product including instructions stored on a non-transitory processor-readable media and executing in a particular processor, including a decentralized distributed computer program executable in any one of a plurality of processors, including the particular processor, including a computing device configured to access non-transitory processor-readable media having instructions that, when executed by the computing device cause the computing device to receive, over at least one communication network from each of a plurality of user computing devices operated by each of a plurality of users, electronic health records (EHR) respectively representing information from the plurality of user's electronic medical records, procured from the user's health care provider or the user's electronic health records (EHR) partners.

Embodiments may also include providing a physical representation on a display, including the user's various organs and body parts, represented in a color-coded system reporting the related electronic health records (EHR) data representing a plurality of medical records laboratory results. Embodiments may also include mapping, on the display of the user's whole-body avatar, the user's electronic health records (EHR) data into a plurality of visual representations including a Severity Risk Index (SRI) representing the risk of a plurality of ailments mapped over the user's plurality of organs and body parts. Embodiments may also include providing, based on a function of the user's electronic health records (EHR) data, the Severity Risk Index (SRI) of the plurality of medical records, and the medical records laboratory results, the user's customized overall health score.

In some embodiments, the user's whole-body avatar may be created by using an image-capturing device or picture file uploaded to the communication network, which may be aggregated with the electronic health records (EHR) data. In some embodiments, the user's electronic health records (EHR) provide further additional details about the Severity Risk Index (SRI) through a clickable navigation link to data attached to the visual representation.

In some embodiments, the processor may be configured to execute instructions that cause the processor to provide a history of the customized overall health score. In some embodiments, the processor may be configured to execute instructions that cause the processor to provide a plurality of score drivers. In some embodiments, the score drivers may include factors representing user-controlled parameters, activities, behaviors, and lifestyles that affect the overall health score may be derived. In some embodiments, the electronic health records (EHR) include information representing at least one of: name, DNA, birthdate, gender, age, height, weight, heart rate, blood pressure, blood type, and blood glucose.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A computer-program product tangibly embodied in a non-transitory computer-readable recording medium in an electronic device for retrieving electronic health records (EHRs) of a patient, the non-transitory computer-readable recording medium storing one or more programs which when executed by a hardware processor cause the non-transitory recording medium to perform steps comprising:
requesting a legally compliant permission from a patient to access a plurality of EHRs of the patient from a plurality of separate electronic storage devices storing at least one of the EHRs of the patient in a non-transitory computer-readable recording medium;
receiving an access request from the patient via a natural language interface to retrieve the EHRs of the patient from the separate electronic storage devices;
processing the access request using a generative artificial intelligence (AI) model having a unique licensing number assigned thereto;
generating via the AI model a structured access request query; and
sending via a communication network the structured access request query with the legally compliant permission to the separate electronic storage devices and thereby retrieving the EHRs.

2. The computer-program product of claim 1, wherein the non-transitory computer-readable recording medium stores one or more programs which when executed by a hardware processor cause the non-transitory recording medium to perform steps comprising:
communicating via the AI model with the communication network; and
providing answers via the AI model to any structured access queries necessary to gain permission to the separate electronic storage devices including at least one of: yes/no answers to verification queries, passwords, alphanumeric user identifications, and access codes.

3. The computer-program product of claim 1, wherein the unique licensing number is a ten-digit National Provider Identifier (NPI) number.

4. The computer-program product of claim 1, wherein the legally compliant permission includes only a name including a current name and any previous names, address information, and a birth date of the patient.

5. The computer-program product of claim 1, wherein the non-transitory computer-readable recording medium stores one or more programs which when executed by a hardware processor cause the non-transitory recording medium to perform steps comprising generating a natural language response summary summarizing the EHRs using the AI model.

6. The computer-program product of claim 5, wherein the non-transitory computer-readable recording medium stores one or more programs which when executed by a hardware processor cause the non-transitory recording medium to perform steps comprising:
receiving an information request from the patient via the natural language interface relating to any portion of the natural language response summary; and
processing the information request using the AI model and providing a reply containing at least one of: health care information, lifestyle information, and suggested actions to perform including performing at-home medical treatments and contacting a medical professional for treatment.

7. The computer-program product of claim 6, wherein the non-transitory computer-readable recording medium stores one or more programs which when executed by a hardware processor cause the non-transitory recording medium to perform steps comprising monitoring and auditing the natural language response summary for accuracy in relation to the information in the EHRs.

8. The computer-program product of claim 1, wherein the non-transitory computer-readable recording medium stores one or more programs which when executed by a hardware processor cause the non-transitory recording medium to perform steps comprising:
requesting legally compliant permission from the patient to access recorded physiological functions from at least one personal electronic health device of the patient storing at least one of the recorded physiological functions of the patient in a non-transitory computer-readable recording medium;
sending via the AI model the legally compliant permission to the at least one personal electronic health device;
generating via the AI model a structured access request query for the recorded physiological functions; and
sending via the AI model the structured access request query to the at least one personal electronic health device and thereby retrieving the recorded physiological functions.

9. The computer-program product of claim 1, wherein the non-transitory computer-readable recording medium stores one or more programs which when executed by a hardware processor cause the non-transitory recording medium to perform steps comprising combining and evaluating the EHRs and the recorded physiological functions and generating a natural language overall health report of the patient using the AI model containing at least one of:
medical status of patient organs and body parts;
comparison of the patient to other patients in a general patient database having similar personal characteristics including age, medical history, lifestyle habits, or genetic information based on relevant and anonymized datasets;
a list of medical personnel to assist with patient health conditions;
a list of medical organizations to assist with patient health conditions;
a list of medical services or products to assist with patient health conditions;
a list of coaches or advisers to assist with patient health conditions;
a list of support groups or networks having persons with similar patient health conditions to assist with patient health conditions; and
a list of non-professional personal contacts to assist with patient health conditions.

10. The computer-program product of claim 1, wherein the non-transitory computer-readable recording medium stores one or more programs which when executed by a hardware processor cause the non-transitory recording medium to perform steps comprising:
generating an image of a human body with the predetermined organs and body parts represented on the image;
analyzing the EHRs and assigning selected data to a respective one of predetermined organs and body parts of the patient;
mapping the selected data to a respective one of the predetermined organs and body parts; and
presenting the image of the human body with the selected data adjacent the predetermined organs and body parts on the personal electronic device of the patient.

11. The computer-program product of claim 1, wherein the non-transitory computer-readable recording medium stores one or more programs which when executed by a hardware processor cause the non-transitory recording medium to perform steps comprising:
analyzing the EHRs and creating an overall health score based for the patient represented by a numerical value in a predetermined range using health risk factors to adjust the overall health score; and
presenting the overall health score on the personal electronic device of the patient.

12. The computer-program product of claim 2, wherein:
the unique licensing number is a ten-digit National Provider Identifier (NPI) number; and
the legally compliant permission includes only a name including a current name and any previous names, address information, and a birth date of the patient.

13. The computer-program product of claim 12, wherein the non-transitory computer-readable recording medium stores one or more programs which when executed by a hardware processor cause the non-transitory recording medium to perform steps comprising:
generating a natural language response summary summarizing the EHRs using the AI model;
receiving an information request from the patient via the natural language interface relating to any portion of the natural language response summary;
processing the information request using the AI model and providing a reply containing at least one of: health care information, lifestyle information, and suggested actions to perform including performing at-home medical treatments and contacting a medical professional for treatment;
monitoring and auditing the natural language response summary for accuracy in relation to the information in the EHRs;
requesting legally compliant permission from the patient to access recorded physiological functions from at least one personal electronic health device of the patient storing at least one of the recorded physiological functions of the patient in a non-transitory computer-readable recording medium;
sending via the AI model the legally compliant permission to the at least one personal electronic health device;
generating via the AI model a structured access request query for the recorded physiological functions;
sending via the AI model the structured access request query to the at least one personal electronic health device and thereby retrieving the recorded physiological functions;
combining and evaluating the EHRs and the recorded physiological functions and generating a natural language overall health report of the patient using the AI model containing at least one of:
medical status of patient organs and body parts;
comparison of the patient to other patients in a general patient database having similar personal characteristics;
a list of medical personnel to assist with patient health conditions;
a list of medical organizations to assist with patient health conditions;
a list of medical services or products to assist with patient health conditions;
a list of coaches or advisers to assist with patient health conditions;
a list of support groups or networks having persons with similar patient health conditions to assist with patient health conditions;
a list of non-professional personal contacts to assist with patient health conditions;
generating an image of a human body with the predetermined organs and body parts represented on the image;
analyzing the EHRs and assigning selected data to a respective one of predetermined organs and body parts of the patient;
mapping the selected data to a respective one of the predetermined organs and body parts;
presenting the image of the human body with the selected data adjacent the predetermined organs and body parts on the personal electronic device of the patient;
analyzing the EHRs and creating an overall health score based for the patient represented by a numerical value in a predetermined range using health risk factors to adjust the overall health score; and
presenting the overall health score on the personal electronic device of the patient.

14. A method of retrieving electronic health records (EHRs) of a patient, the method comprising the steps of:
requesting, via the computer-program product tangibly embodied in a non-transitory computer-readable recording medium in an electronic device for retrieving electronic health records (EHRs) of a patient, legally compliant permission from a patient to access a plurality of EHRs of the patient from a plurality of separate electronic storage devices storing at least one of the EHRs of the patient in a non-transitory computer-readable recording medium;
receiving an access request from the patient via a natural language interface to retrieve the EHRs of the patient from the separate electronic storage devices;
processing the access request using a generative artificial intelligence (AI) model having a unique licensing number assigned thereto;
generating via the AI model a structured access request query; and
sending via a communication network the structured access request query with the legally compliant permission to the separate electronic storage devices and thereby retrieving the EHRs.

15. The method of claim 14, further comprising:
communicating via the AI model with the communication network; and
providing answers via the AI model to any structured access queries necessary to gain permission to the separate electronic storage devices including at least one of: yes/no answers to verification queries, passwords, alphanumeric user identifications, and access codes.

16. The method of claim 14, wherein the unique licensing number is a ten-digit National Provider Identifier (NPI) number.

17. The method of claim 14, wherein the legally compliant permission includes only a name including a current name and any previous names, address information, and a birth date of the patient.

18. The method of claim 14, further comprising:
generating a natural language response summary summarizing the EHRs using the AI model;
receiving an information request from the patient via the natural language interface relating to any portion of the natural language response summary;
processing the information request using the AI model and providing a reply containing at least one of: health care information, lifestyle information, and suggested actions to perform including performing at-home medical treatments and contacting a medical professional for treatment; and
repeating the steps of receiving and processing to create a dynamic feedback loop and thereby further customizing using the AI model the reply for the patient.

19. The method of claim 15, wherein the unique licensing number is a ten-digit National Provider Identifier (NPI) number, the legally compliant permission includes only a name including a current name and any previous names, address information, and a birth date of the patient, and the method further comprises:
generating a natural language response summary summarizing the EHRs using the AI model;
receiving an information request from the patient via the natural language interface relating to any portion of the natural language response summary;
processing the information request using the AI model and providing a reply containing at least one of: health care information, lifestyle information, and suggested actions to perform including performing at-home medical treatments and contacting a medical professional for treatment;
monitoring and auditing the natural language response summary for accuracy in relation to the information in the EHRs;
requesting legally compliant permission from the patient to access recorded physiological functions from at least one personal electronic health device of the patient storing at least one of the recorded physiological functions of the patient in a non-transitory computer-readable recording medium;
sending via the AI model the legally compliant permission to the at least one personal electronic health device;
generating via the AI model a structured access request query for the recorded physiological functions;
sending via the AI model the structured access request query to the at least one personal electronic health device and thereby retrieving the recorded physiological functions;
combining and evaluating the EHRs and the recorded physiological functions and generating a natural language overall health report of the patient using the AI model containing at least one of:
medical status of patient organs and body parts;
comparison of the patient to other patients in a general patient database having similar personal characteristics;
a list of medical personnel to assist with patient health conditions;
a list of medical organizations to assist with patient health conditions;
a list of medical services or products to assist with patient health conditions;
a list of coaches or advisers to assist with patient health conditions;
a list of support groups or networks having persons with similar patient health conditions to assist with patient health conditions;
a list of non-professional personal contacts to assist with patient health conditions;
generating an image of a human body with the predetermined organs and body parts represented on the image;
analyzing the EHRs and assigning selected data to a respective one of predetermined organs and body parts of the patient;
mapping the selected data to a respective one of the predetermined organs and body parts;
presenting the image of the human body with the selected data adjacent the predetermined organs and body parts on the personal electronic device of the patient;
analyzing the EHRs and creating an overall health score based for the patient represented by a numerical value in a predetermined range using health risk factors to adjust the overall health score; and
presenting the overall health score on the personal electronic device of the patient.

20. An electronic health record (EHR) retrieval system for performing the method of claim 14, the EHR retrieval system comprising:
a computer-program product, the computer-program product being tangibly embodied in a non-transitory computer-readable recording medium in an electronic device for retrieving electronic health records (EHRs) of a patient, the non-transitory computer-readable recording medium storing one or more programs which when executed by a hardware processor cause the non-transitory recording medium to perform steps comprising:
requesting legally compliant permission from a patient to access a plurality of EHRs of the patient from a plurality of separate electronic storage devices storing at least one of the EHRs of the patient in a non-transitory computer-readable recording medium;
receiving an access request from the patient via a natural language interface to retrieve the EHRs of the patient from the separate electronic storage devices;
processing the access request using a generative artificial intelligence (AI) model having a unique licensing number assigned thereto;
generating via the AI model a structured access request query; and
sending via a communication network the structured access request query with the legally compliant permission to the separate electronic storage devices and thereby retrieving the EHRs;
a personal electronic device, the personal electronic device being configured to display an interface for a user to interact with the computer-program product; and
a plurality of separate electronic storage devices, the separate electronic storage devices being configured to store EHRs in a non-transitory computer-readable recording medium.
